(19) United States
(12) Patent Application Publication
(10) Pub. No.: US 2025/0245893 A1
(43) Pub. Date: Jul. 31, 2025

(54) APPARATUS FOR MEDICAL GRAPHICS EDITION

(71) Applicants: Fu-Ming WANG, Taipei City (TW); Yu-Te WU, Taipei City (TW); Wan-Yuo GUO, Taipei City (TW)

(72) Inventor: Fu-Ming WANG, Taipei City (TW)

(21) Appl. No.: 19/033,718

(22) Filed: Jan. 22, 2025

(30) Foreign Application Priority Data
Jan. 29, 2024 (TW) 113103411

(51) Int. Cl. G06T 11/60; G06T 3/40; G16H 30/40

(52) U.S. Cl. CPC G06T 11/60; G06T 3/40; G16H 30/40; G06T 2200/24; G06T 2210/41

(57) ABSTRACT

An apparatus for medical graphics edition includes a display device, an electronic calculator, and a medical graphics editor. The display device displays at least one medical image, and the medical graphics editor is executed by the electronic calculator. The medical graphics editor includes a shaping tool, which is used to shape a contour in the medical image. The shaping tool includes at least one shaping pattern and a shaping vector pattern. The shaping vector pattern is combined with the shaping pattern to form a pattern of the shaping tool. The shaping tool is to cause the contour, based on the shaping pattern, to have a shape change in a proportion in the direction indicated by the shaping vector pattern. In addition, the medical graphics editor further includes a scaling tool.

### CROSS REFERENCE TO RELATED APPLICATIONS

This Non-provisional application claims priority under 35 U.S.C. § 119(a) on Patent Application No(s). 113103411 filed in Taiwan, Republic of China on Jan. 29, 2024, the entire contents of which are hereby incorporated by reference.

### BACKGROUND

### Technology Field

The present disclosure relates to an apparatus for graphics editor and, in particular, to an apparatus for medical graphics editor that can be used to edit various medical images.

### Description of Related Art

Medical imaging technology has made a great progress in recent years. The medical imaging technologies such as MRI, MRA, and CT have been widely used in medical detection. After obtaining various medical detection images, the medical units usually have to use a medical image browser to view the medical detection images (medical images) of patients, and then use the editor in the browser or use an independent editor to manually mark the patient's lesion with a contour on the patient's medical image, so that the doctor can evaluate the patient's condition and further make the subsequent medical treatment plan based on the marked lesion.

With the rapid development of medical AI technology, the step of marking lesions on medical images has gradually evolved from manually marking to AI automatic marking. However, both of the manually marking and the AI automatic marking may encounter the problem of incorrect marking or poor marking quality. To solve this problem, the medical personnel must use the modifying tool provided by the medical graphics editor to correct the contour.

For example, as shown in FIG. 1, the user can use the modifying tool to mark a plurality of marking points 90 in the area near the segment XY of the contour 9 to be modified, and then link the plurality of marking points 90 to form a modified segment 91 (the dotted line as shown in FIG. 2). In one case, when the needed modified segment is longer, the user has to mark more marking points 90 to complete the modification. In another case, when the shape of the needed modified segment has a relatively special shape, the user may not accurately represent the needed modified segment by marking a plurality of marking points 90.

In addition to the above-mentioned modifying tool, there are many different types of modifying tools in the conventional art for modifying the medical images. In addition, the conventional medical graphics editor usually has a scaling tool that allows the user to zoom in or out the entire or part of the contour. However, there is a common problem in all conventional scaling tools of the medical graphics editors, which is that the contour cannot be easily and quickly modified to fit the actual lesions of patients. That is, the user may spend a relatively long time to modify the contour marking actual lesions of patients. Similarly, this problem also exists in the case of using the scaling tool to zoom in or out the entire or part of the contour.

Therefore, it is desired to provide a solution that can easily, quickly and actually modify the contour in the medical image.

### SUMMARY

In view of the foregoing, an objective of this disclosure is to provide an apparatus for medical graphics edition that can allow the user to easily, quickly and actually modify the contour in the medical image.

To achieve the above, the present disclosure provides an apparatus for medical graphics edition, which includes a display device, an electronic calculator, and a medical graphics editor. The display device displays a cursor and at least a medical image formed with a contour. The medical graphics editor is executed by the electronic calculator, and the medical graphics editor includes a shaping tool. The shaping tool is used to shape the contour in the medical image. The shaping tool includes at least one shaping pattern and a shaping vector pattern, and one end of the shaping vector pattern is combined with the shaping pattern to form a pattern of the shaping tool. The shaping tool is configured to cause a part of the contour, based on the shaping pattern, to have a shape change in a proportion in a direction indicated by the shaping vector pattern.

In one embodiment, the shaping tool functions based on the cursor.

In one embodiment, another end of the shaping vector pattern is connected to two end points of the shaping pattern to form a first shaping part adjusting line and a second shaping part adjusting line, the first shaping part adjusting line and the second shaping part adjusting line individually cross with the contour at two intersection points, respectively, and the part of the contour between the two intersection points has the shape change based on a shape of the shaping pattern.

In one embodiment, the shape of the shaping pattern is changed by adjusting an included angle between the first shaping part adjusting line and the second shaping part adjusting line.

In one embodiment, a pattern shape adjusting symbol is configured at an intersection of the shaping vector pattern and the shaping pattern, and the shape of the shaping pattern is changed by adjusting a position of the pattern shape adjusting symbol.

In one embodiment, the shaping vector pattern is configured with a shaping position designating symbol, the shaping position designating symbol moves in an extension direction of the shaping vector pattern, and the part of the contour having the shape change passes through a point designated by the shaping position designating symbol.

In one embodiment, the part of the contour is caused to have the shape change by using the cursor to click the pattern of the shaping tool.

In one embodiment, the shaping pattern has a shape of a curve, an arrow, a linear line, or a sawtooth.

Since the shaping tool of the apparatus for medical image edition of the present disclosure can change the shaping pattern of the shaping tool according to the modification requirement of the contour, the contour can be modified more accurately. Moreover, because the user can directly select different shaping patterns to achieve the modification, he or she can easily, quickly, and accurately modify the contour in the medical image. In other words, the apparatus for medical image edition of the present disclosure can reduce the time of marking the target and thus improve the effect of precise treatment.

In one embodiment, the medical graphics editor can further include a scaling tool, which includes a scaling pattern and a scaling vector pattern. The scaling vector pattern is combined with the scaling pattern to form a pattern of the scaling tool. The scaling tool is configured to cause a part or all of the contour, based on a shape of the original contour, to have a scale change in a proportion in a direction indicated by the scaling vector pattern.

To achieve the above, this disclosure also provides an apparatus for medical graphics edition, which includes a display device, an electronic calculator, and a medical graphics editor. The display device displays a cursor and at least a medical image formed with a contour. The medical graphics editor is executed by the electronic calculator, and the medical graphics editor includes a scaling tool. The scaling tool is used to zoom in or zoom out the contour in the medical image. The scaling tool includes at least one scaling pattern and a scaling vector pattern, and the scaling vector pattern is combined with the scaling pattern to form a pattern of the scaling tool. The scaling tool is configured to cause a part or all of the contour, based on a shape of the original contour, to have a scale change in a proportion in a direction indicated by the scaling vector pattern.

In one embodiment, the scaling pattern has a shape of an enclosed curve.

In one embodiment, the scaling vector pattern is a zoom-in vector pattern or a zoom-out vector pattern.

In one embodiment, the zoom-in vector pattern includes a first zoom-in vector pattern and a second zoom-in vector pattern pointing in a direction from a circle center to a circumference, and the zoom-out vector pattern includes a first zoom-out vector pattern and a second zoom-out vector pattern pointing in a direction from a circumference to a circle center.

In one embodiment, a first zoom-in part adjusting line is formed in an extension direction of the first zoom-in vector pattern, a second zoom-in part adjusting line is formed in an extension direction of the second zoom-in vector pattern, the first zoom-in part adjusting line and the second zoom-in part adjusting line individually cross with the contour at two intersection points, respectively, and the part of the contour between the two intersection points is zoomed in in a proportion based on the shape of the original contour.

In one embodiment, a first zoom-out part adjusting line is formed in an extension direction of the first zoom-out vector pattern, a second zoom-out part adjusting line is formed in an extension direction of the second zoom-out vector pattern, the first zoom-out part adjusting line and the second zoom-out part adjusting line individually cross with the contour at two intersection points, respectively, and the part of the contour between the two intersection points is zoomed out in a proportion based on the shape of the original contour.

In one embodiment, when the first zoom-in vector pattern and the second zoom-in vector pattern of the zoom-in vector pattern are overlapped with each other, the scaling tool zooms in the contour in all directions.

In one embodiment, when the first zoom-out vector pattern and the second zoom-out vector pattern of the zoom-out vector pattern are overlapped with each other, the scaling tool zooms out the contour in all directions.

In one embodiment, the zoom-in vector pattern further includes a third zoom-in vector pattern pointing in a direction from a circle center to a circumference, and the zoom-out vector pattern further includes a third zoom-out vector pattern pointing in a direction from a circumference to a circle center.

In one embodiment, the third zoom-in vector pattern is configured with a zoom-in position designating symbol, the zoom-in position designating symbol moves in an extension direction of the third zoom-in vector pattern, and the part or all of the contour being zoomed in passes through a point designated by the zoom-in position designating symbol.

In one embodiment, the third zoom-out vector pattern is configured with a zoom-out position designating symbol, the zoom-out position designating symbol moves in an extension direction of the third zoom-out vector pattern, and the part or all of the contour being zoomed in passes through a point designated by the zoom-out position designating symbol.

Since the scaling tool of the apparatus for medical image edition of the present disclosure can cause a part or all of the contour, based on a shape of the original contour, to have a scale change in a proportion in a direction indicated by the scaling vector pattern, the user can easily and quickly modify the contour in the medical image. Moreover, the scaling tool and the shaping tool can operate together to more actually modify the contour in the medical image.

### BRIEF DESCRIPTION OF THE DRAWINGS

The disclosure will become more fully understood from the detailed description and accompanying drawings, which are given for illustration only, and thus are not limitative of the present disclosure, and wherein:

FIG. 1 is a schematic diagram showing the conventional modification of the contour in the medical image;

FIG. 2 is a schematic diagram showing the modified contour in the medical image of FIG. 1;

FIG. 3 is a schematic diagram showing an apparatus for medical image edition according to an embodiment of this disclosure;

FIG. 4 is a schematic diagram showing a shaping tool of the apparatus for medical image edition according to an embodiment of this disclosure;

FIG. 5 is a schematic diagram showing another shaping tool of the apparatus for medical image edition according to an embodiment of this disclosure;

FIG. 6A is a schematic diagram showing the procedure of shaping the contour in one direction by using the shaping tool of the apparatus for medical image edition according to an embodiment of this disclosure;

FIG. 6B is a schematic diagram showing the procedure of shaping the contour in another direction by using the shaping tool of the apparatus for medical image edition according to an embodiment of this disclosure;

FIG. 7 is a schematic diagram showing the procedure of shaping the contour by using a shaping position designating symbol of the shaping tool of the apparatus for medical image edition according to an embodiment of this disclosure;

FIG. 8 is a schematic diagram showing the procedure of changing the shaping pattern by adjusting the included angle between the first shaping part adjusting line and the second shaping part adjusting line;

FIG. 9 is another schematic diagram showing the procedure of changing the shaping pattern by adjusting the included angle between the first shaping part adjusting line and the second shaping part adjusting line;

FIG. 10 is a schematic diagram showing the procedure of shaping the contour by adjusting a pattern shape adjusting symbol of the shaping tool of the apparatus for medical image edition according to an embodiment of this disclosure;

FIG. 11 is another schematic diagram showing the procedure of shaping the contour by adjusting a pattern shape adjusting symbol of the shaping tool of the apparatus for medical image edition according to an embodiment of this disclosure;

FIG. 12A is a schematic diagram showing a scaling tool of the apparatus for medical image edition according to an embodiment of this disclosure;

FIG. 12B is a schematic diagram showing another scaling tool of the apparatus for medical image edition according to an embodiment of this disclosure;

FIG. 13A is a schematic diagram showing the procedure of zooming in the contour in one direction while the scaling vector pattern is a zoom-in vector pattern;

FIG. 13B is a schematic diagram showing the procedure of zooming out the contour in one direction while the scaling vector pattern is a zoom-out vector pattern;

FIG. 14 is a schematic diagram showing the procedure of zooming in the contour while the scaling vector pattern is a zoom-in vector pattern and the zoom-in position designating symbol is moved in one direction;

FIG. 15A is a schematic diagram showing the scaling tool when the first zoom-in vector pattern and the second zoom-in vector pattern are overlapped with each other;

FIG. 15B is a schematic diagram showing the scaling tool when the first zoom-out vector pattern and the second zoom-out vector pattern are overlapped with each other;

FIG. 16A is a schematic diagram showing the procedure that the scaling tool zooms in the contour in all directions when the first zoom-in vector pattern and the second zoom-in vector pattern are overlapped with each other; and

FIG. 16B is a schematic diagram showing the procedure that the scaling tool zooms out the contour in all directions when the first zoom-out vector pattern and the second zoom-out vector pattern are overlapped with each other.

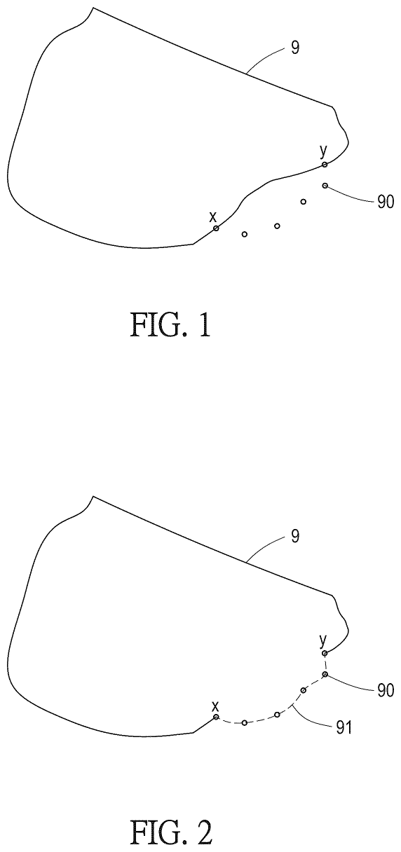

### DETAILED DESCRIPTION OF THE DISCLOSURE

The present disclosure will be apparent from the following detailed description, which proceeds with reference to the accompanying drawings, wherein the same references relate to the same elements.

In order to avoid redundancy, before describing the specific embodiments of the present disclosure, the relevant terms used in the present disclosure are defined as follows:

The term “electronic calculator” refers to all kinds of computers with CPU or GPU including, for example but not limited to, desktop computers, laptops, tablets, mobile phones, or the likes. The term “medical graphics editor” refers to an editor that can edit various kinds of medical image formats such as DICOM, RTSS, Nifti, or Analyze.

In addition, in the embodiments of this disclosure, the term “symbol” or “pattern” that has similar visual effect can be considered as the “symbol” or “pattern” with the same function.

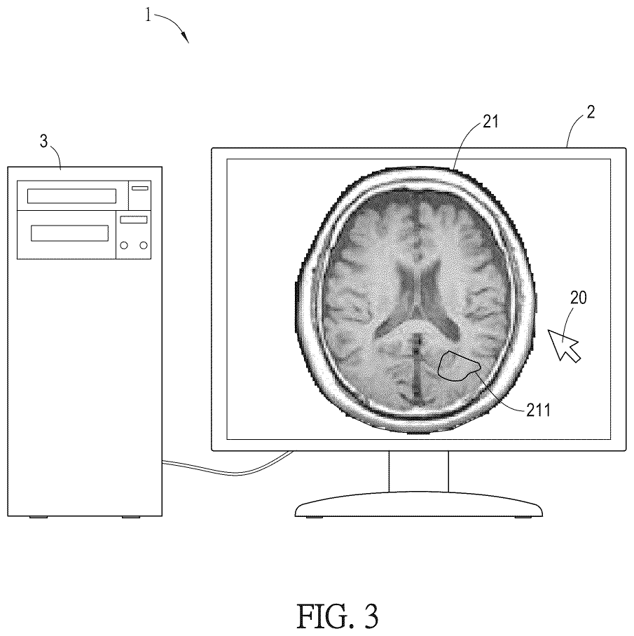

As shown in FIG. 3, the apparatus 1 for medical image edition of this disclosure includes a display device 2, and electronic calculator 3 and a medical graphics editor. The display device 2 displays a cursor 20 and at least a medical image 21 formed with a contour 211. The medical graphics editor is executed by the electronic calculator 3.

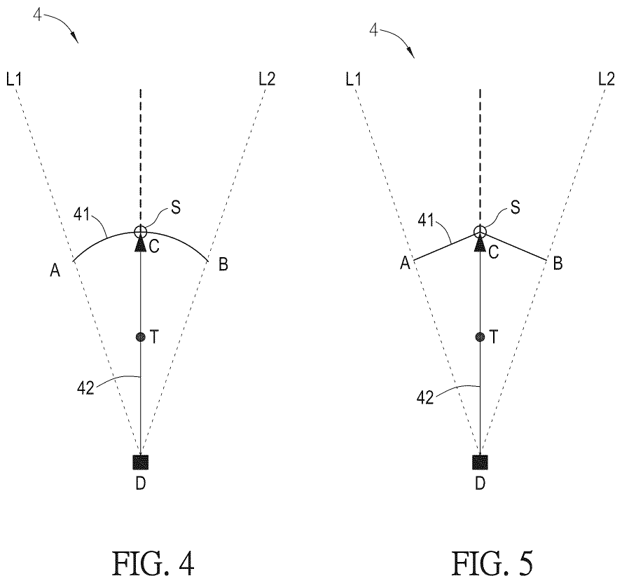

Referring to FIG. 4 or FIG. 5, the medical graphics editor includes a shaping tool 4, which is used to shape the contour 211 in the medical image 21. The shaping tool 4 includes at least one shaping pattern 41 and a shaping vector pattern 42. One end point C of the shaping vector pattern 42 is combined with the shaping pattern 41 to form a pattern of the shaping tool 4. The shaping tool 4 is configured to cause a part of the contour 211, based on the shaping pattern 41, to have a shape change in a proportion in a direction indicated by the shaping vector pattern 42.

In this embodiment, the shaping pattern 41 may have a shape of a curve (see FIG. 4), an arrow (see FIG. 5), a linear line (not shown), or a sawtooth (not shown). The other end point D of the shaping vector pattern 42 is connected to the two end points A and B of the shaping pattern 41 respectively to form a first shaping part adjusting line L1 and a second shaping part adjusting line L2.

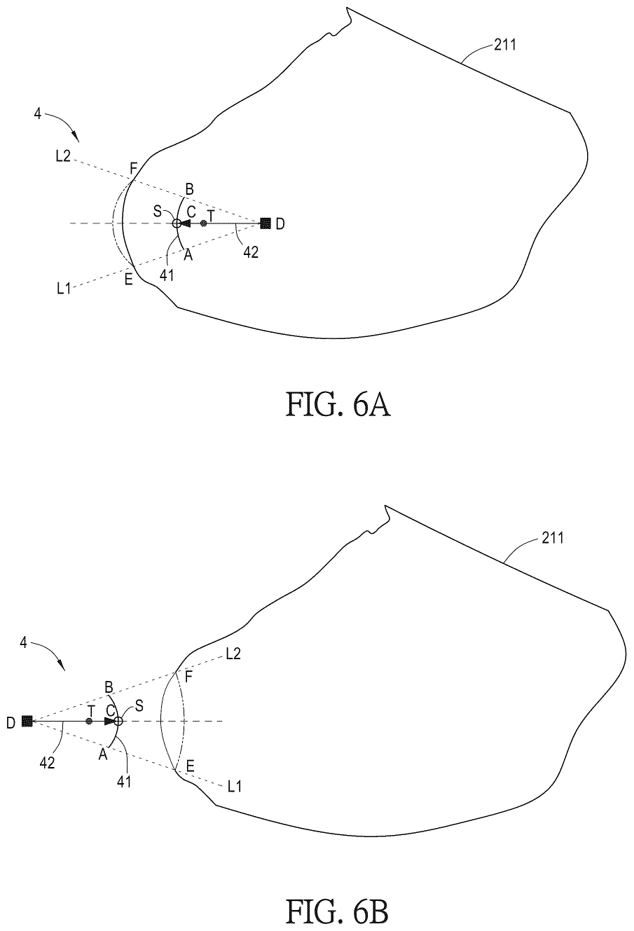

Referring to FIG. 6A or FIG. 6B, the first shaping part adjusting line L1 crosses with the contour 211 at the intersection point E, and the second shaping part adjusting line L2 crosses with the contour 211 at the intersection point F. The part of the contour 211 between the two intersection points E and F has a shape change based on a shape of the shaping pattern 41. To be noted, as shown in FIGS. 6A and 6B, the user may control the cursor 20 to move the end point D around the end point C so as to change the direction of the shaping vector pattern 42 of the shaping tool 4. Moreover, the user may also control the cursor 20 to move the position of the entire shaping tool 4.

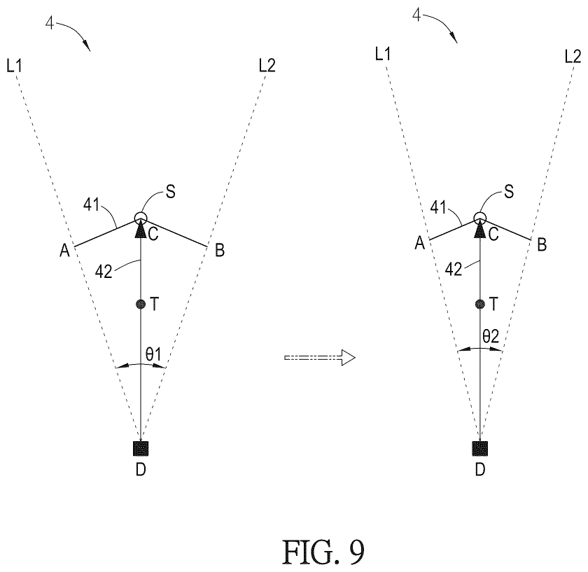

In this embodiment, the shape of the shaping pattern 41 cab be changed by adjusting an included angle between the first shaping part adjusting line L1 and the second shaping part adjusting line L2. As shown in FIG. 8, the shaping pattern 41 has a shape of a curve. When the included angle between the first shaping part adjusting line L1 and the second shaping part adjusting line L2 is changed from the angle θ1 to the angle θ2, the shape of the shaping pattern 41 can be changed from a longer curve pattern (see the left part of FIG. 8) to a shorter curve pattern (see the right part of FIG. 8). In another case, as shown in FIG. 9, the shaping pattern 41 has a shape of an arrow. When the included angle between the first shaping part adjusting line L1 and the second shaping part adjusting line L2 is changed from the angle θ1 to the angle θ2, the shape of the shaping pattern 41 can be changed from a larger arrow pattern (see the left part of FIG. 9) to a smaller arrow pattern (see the right part of FIG. 9).

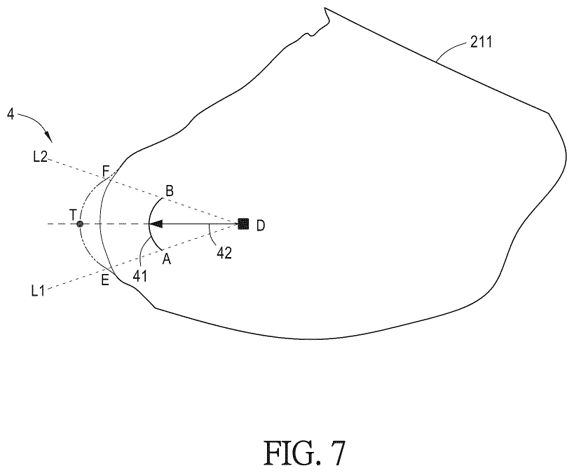

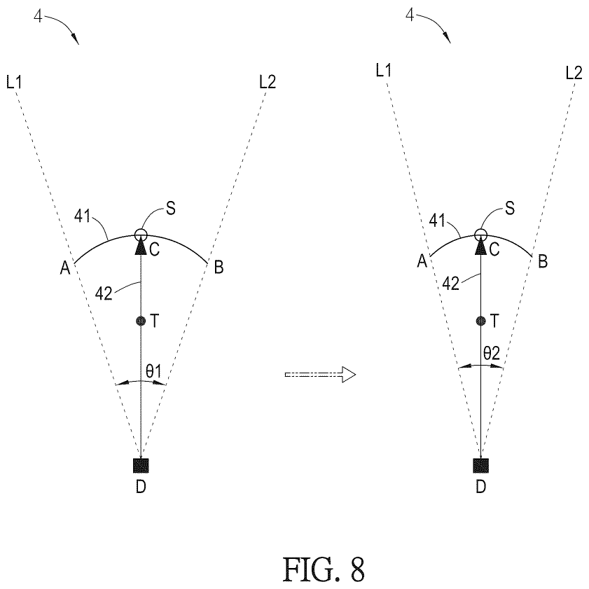

Referring to FIG. 4 or FIG. 5 again, the shaping vector pattern 42 is configured with a shaping position designating symbol T. Referring to FIG. 7, the shaping position designating symbol T can move in an extension direction of the shaping vector pattern 42, and the part segment EF of the contour 211 having the shape change passes through a point designated by the shaping position designating symbol T.

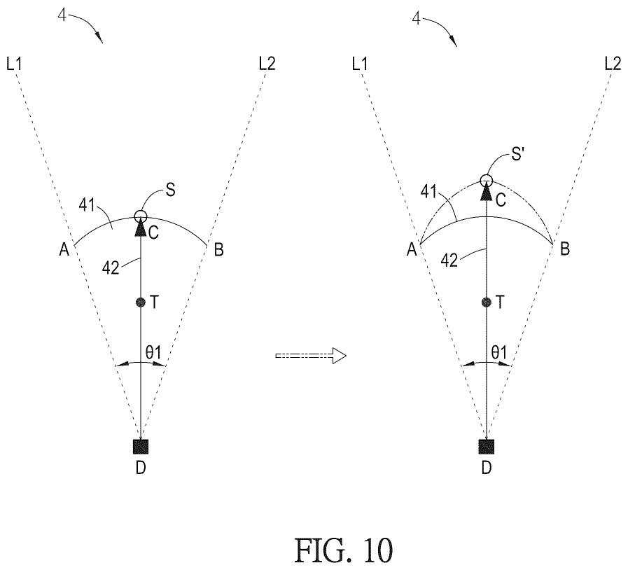

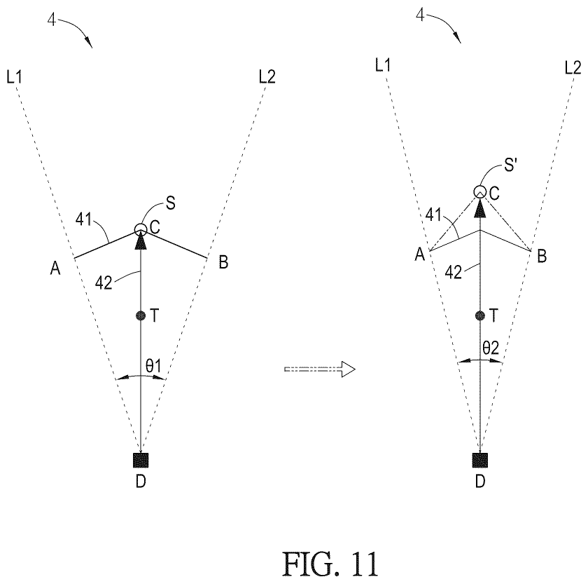

In addition to adjusting the shape of the shaping pattern 41 by adjusting the included angle between the first shaping part adjusting line L1 and the second shaping part adjusting line L2, the present disclosure may provide a pattern shape adjusting symbol S at an intersection of the shaping vector pattern 42 and the shaping pattern 41, and the shape of the shaping pattern 41 can be changed by adjusting a position of the pattern shape adjusting symbol S (e.g. from S to S′). As shown in FIG. 10, the shaping pattern 41 has a shape of a curve. When the position of the pattern shape adjusting symbol S is moved upwardly (see the right part of FIG. 10), the shape of the shaping pattern 41 can be changed from a curve pattern with a larger curvature radius to a curve pattern with a smaller curvature radius. In another case, as shown in FIG. 11, the shaping pattern 41 has a shape of an arrow. When the position of the pattern shape adjusting symbol S is moved upwardly (see the right part of FIG. 11), the shape of the shaping pattern 41 can be changed from an arrow pattern with a smaller sharpness to an arrow pattern with a larger sharpness.

As mentioned above, in the embodiment, after setting the shape of the shaping pattern 41, the user can control the cursor to click the pattern of the shaping tool to cause a part of the contour to have a shape change in a proportion based on the shaping pattern 41. In one case, the proportion of the shape change can be determined based on, for example, the number of clicks. In addition, since the shaping pattern 41 of the shaping tool 4 of the present disclosure can be changed arbitrarily, and the size of the shape thereof can also be adjusted arbitrarily, the user can easily and quickly find the appropriate shaping tool 4 corresponding to various shapes of the contour 211, thereby performing the modification of the contour 211 effectively.

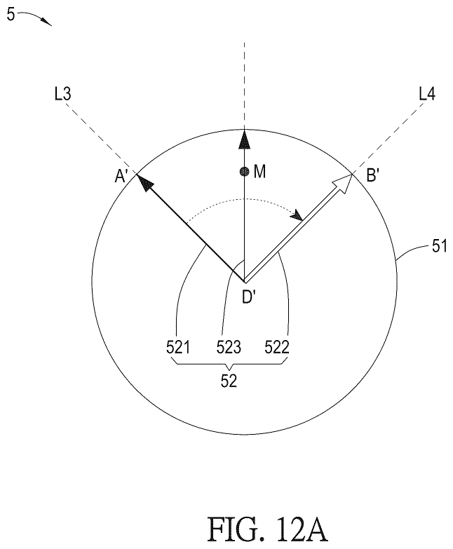

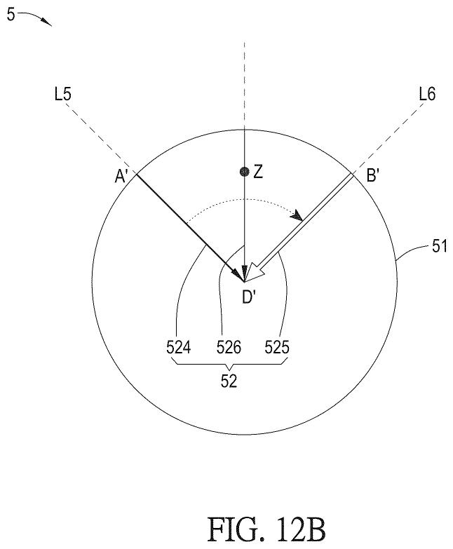

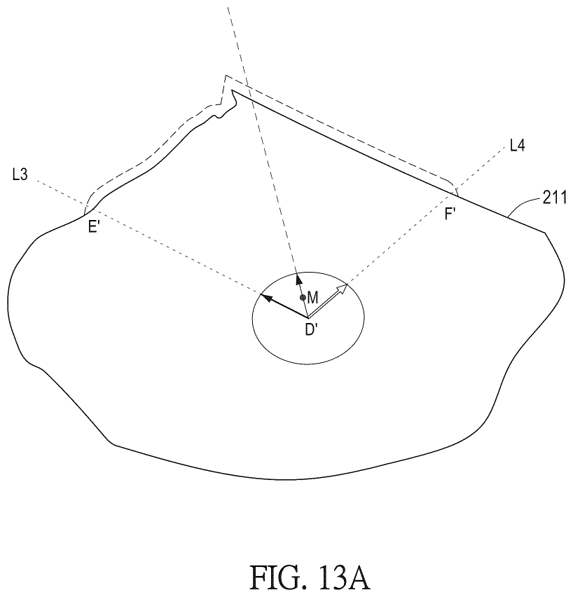

Referring to FIG. 12A or FIG. 12B, the medical graphics editor of this disclosure can further include a scaling tool 5, which includes at least one scaling pattern 51 and a scaling vector pattern 52. The scaling vector pattern 52 is combined with the scaling pattern 51 (e.g. at points A′ and B′) to form a pattern of the scaling tool 5. The scaling tool 5 is configured to cause a part or all of the contour, based on a shape of the original contour, to have a scale change in a proportion in a direction indicated by the scaling vector pattern 52.

Referring to FIG. 12A, the scaling pattern 51 can have a shape of an enclosed curve, such as a circle (with the circle center D′), and the scaling vector pattern 52 is a zoom-in vector pattern. In this embodiment, the zoom-in vector pattern includes a first zoom-in vector pattern 521 and a second zoom-in vector pattern 522 pointing in a direction from the circle center to the circumference of the circle. A first zoom-in part adjusting line L3 is formed in an extension direction of the first zoom-in vector pattern 521, and a second zoom-in part adjusting line L4 is formed in an extension direction of the second zoom-in vector pattern 522.

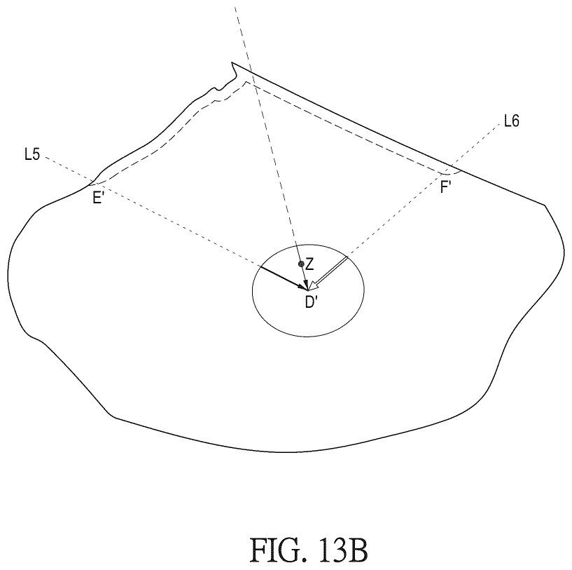

Referring to FIG. 13A, the first zoom-in part adjusting line L3 crosses with the contour at the intersection point E′, and the second zoom-in part adjusting line L4 crosses with the contour at the intersection point F′. The part of the contour between the two intersection points E′ ad F′ can be zoomed in in a proportion based on the shape of the original contour, and the proportion of the zoom-in operation can be determined based on, for example, the number of clicks.

Referring to FIG. 12B, the scaling pattern 51 can have a shape of an enclosed curve, such as a circle (with the circle center D′), and the scaling vector pattern 52 is a zoom-out vector pattern. In this embodiment, the zoom-out vector pattern includes a first zoom-out vector pattern 524 and a second zoom-out vector pattern 525 pointing in a direction from the circumference to the circle center of the circle. A first zoom-out part adjusting line L5 is formed in an extension direction of the first zoom-out vector pattern 524, and a second zoom-out part adjusting line L6 is formed in an extension direction of the second zoom-out vector pattern 525.

Referring to FIG. 13B, the first zoom-out part adjusting line L5 crosses with the contour at the intersection point E′, and the second zoom-out part adjusting line L6 crosses with the contour at the intersection point F′. The part of the contour between the two intersection points E′ ad F′ can be zoomed out in a proportion based on the shape of the original contour, and the proportion of the zoom-out operation can be determined based on, for example, the number of clicks.

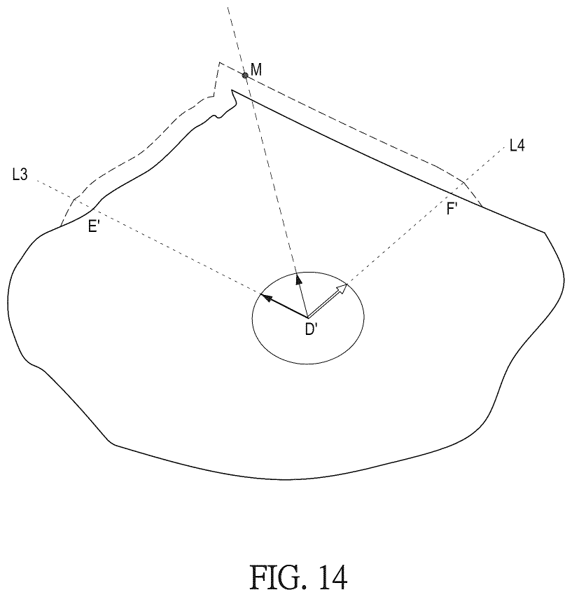

Referring to FIG. 12A or FIG. 12B again, the zoom-in vector pattern further includes a third zoom-in vector pattern 523 pointing in a direction from the circle center to the circumference, and the zoom-out vector pattern further includes a third zoom-out vector pattern 526 pointing in a direction from the circumference to the circle center. As shown in FIG. 14, the third zoom-in vector pattern 523 is configured with a zoom-in position designating symbol M, and the zoom-in position designating symbol M can move in an extension direction of the third zoom-in vector pattern 523. The part or all of the contour 211 being zoomed in passes through a point designated by the zoom-in position designating symbol M. In other words, the user can directly adjust the position of the zoom-in position designating symbol M to zoom in all or part of the contour 211 at one time. In addition, the third zoom-out vector pattern 526 is configured with a zoom-out position designating symbol Z, and the zoom-out position designating symbol Z can move in an extension direction of the third zoom-out vector pattern 526. The part or all of the contour 211 being zoomed out passes through a point designated by the zoom-out position designating symbol Z. In other words, the user can directly adjust the position of the zoom-out position designating symbol Z to zoom out all or part of the contour 211 at one time.

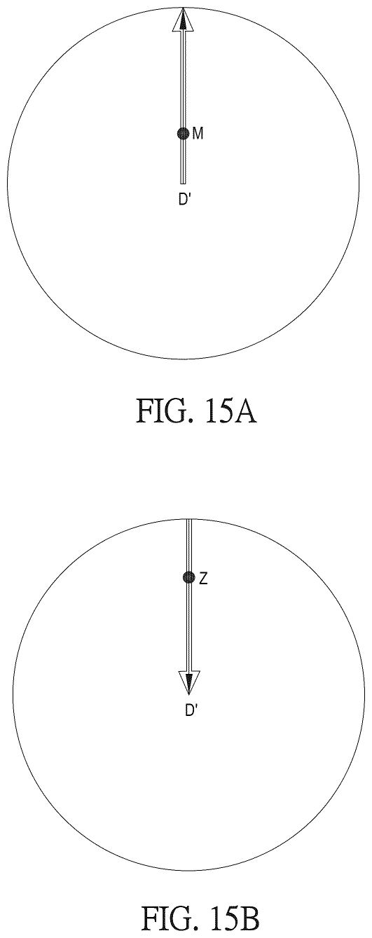

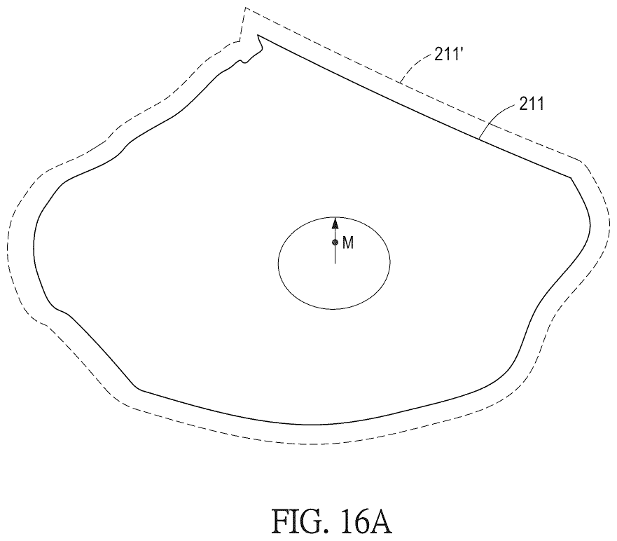

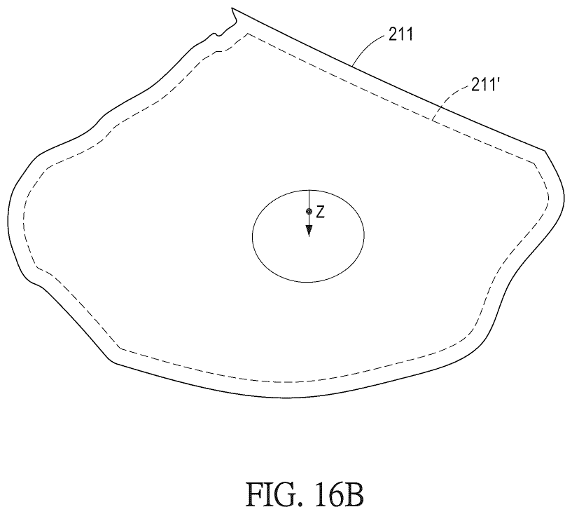

Referring to FIG. 15A, in this embodiment, when the first zoom-in vector pattern 521, the second zoom-in vector pattern 522, and the third zoom-in vector pattern 523 are overlapped with each other, or when the first zoom-in part adjusting line L3 and the second zoom-in part adjusting line LA are overlapped with each other, the scaling tool 5 can zoom in the contour 211 in all directions (in 360 degrees). This shape change of the contour 211 is shown in FIG. 16A (from 211 to 211′). Similarly, as shown in FIG. 15B, when the first zoom-out vector pattern 524, the second zoom-out vector pattern 525, and the third zoom-out vector pattern 526 are overlapped with each other, or when the first zoom-out part adjusting line L5 and the second zoom-out part adjusting line L6 are overlapped with each other, the scaling tool 5 can zoom out the contour 211 in all directions (in 360 degrees). This scale change of the contour 211 is shown in FIG. 16B (from 211 to 211′).

As mentioned above, in the embodiment, after setting the shape of the scaling pattern 51, the user can control the cursor to click the pattern of the scaling tool to cause a part of the contour to have a scale change (zoom in or zoom out) in a proportion based on the shape of the original contour. In one case, the proportion of the scale change can be determined based on, for example, the number of clicks. In addition, since the scaling pattern 51 of the scaling tool 5 of the present disclosure can cause the scale change arbitrarily, the user can easily and quickly modify various shapes of the contour 211. Moreover, if the user uses the scaling tool 5 interactively with the above-mentioned shaping tool 4, the original contour can be modified into a more precise contour more quickly, thereby improving the effect of precise treatment.

In addition, this disclosure also provides an apparatus for medical graphics edition, which includes a display device, an electronic calculator, and a medical graphics editor. The medical graphics editor includes a scaling tool. The detailed descriptions of the scaling tool can be referred to the above-mentioned scaling tool 5, so the descriptions thereof will be omitted. To be noted, the medical graphics editor of this disclosure can be installed in a general medical image browser.

Although the disclosure has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the disclosure.

What is claimed is:

1. An apparatus for medical graphics edition, comprising a display device, an electronic calculator, and a medical graphics editor, wherein the display device displays a cursor and at least a medical image formed with a contour, the medical graphics editor is executed by the electronic calculator, the medical graphics editor comprises a shaping tool, the shaping tool is used to shape the contour in the medical image, and the shaping tool comprises:
at least one shaping pattern; and
a shaping vector pattern, wherein one end of the shaping vector pattern is combined with the shaping pattern to form a pattern of the shaping tool;
wherein, the shaping tool is to cause a part of the contour, based on the shaping pattern, to have a shape change in a proportion in a direction indicated by the shaping vector pattern.

2. The apparatus of claim 1, wherein the shaping tool functions based on the cursor.

3. The apparatus of claim 1, wherein another end of the shaping vector pattern is connected to two end points of the shaping pattern to form a first shaping part adjusting line and a second shaping part adjusting line, the first shaping part adjusting line and the second shaping part adjusting line individually cross with the contour at two intersection points, respectively, and the part of the contour between the two intersection points has the shape change based on a shape of the shaping pattern.

4. The apparatus of claim 3, wherein the shape of the shaping pattern is changed by adjusting an included angle between the first shaping part adjusting line and the second shaping part adjusting line.

5. The apparatus of claim 1, wherein a pattern shape adjusting symbol is configured at an intersection of the shaping vector pattern and the shaping pattern, and the shape of the shaping pattern is changed by adjusting a position of the pattern shape adjusting symbol.

6. The apparatus of claim 1, wherein the shaping vector pattern is configured with a shaping position designating symbol, the shaping position designating symbol moves in an extension direction of the shaping vector pattern, and the part of the contour having the shape change passes through a point designated by the shaping position designating symbol.

7. The apparatus of claim 6, wherein the part of the contour is caused to have the shape change by using the cursor to click the pattern of the shaping tool.

8. The apparatus of claim 1, wherein the shaping pattern has a shape of a curve, an arrow, a linear line, or a sawtooth.

9. An apparatus for medical graphics edition, comprising a display device, an electronic calculator, and a medical graphics editor, wherein the display device displays a cursor and at least a medical image formed with a contour, the medical graphics editor is executed by the electronic calculator, the medical graphics editor comprises a scaling tool, the scaling tool is used to zoom in or zoom out the contour in the medical image, and the scaling tool comprises:
at least one scaling pattern; and
a scaling vector pattern, wherein the scaling vector pattern is combined with the scaling pattern to form a pattern of the scaling tool;
wherein, the scaling tool is to cause a part or all of the contour, based on a shape of the original contour, to have a scale change in a proportion in a direction indicated by the scaling vector pattern.

10. The apparatus of claim 9, wherein the scaling pattern has a shape of an enclosed curve.

11. The apparatus of claim 9, wherein the scaling vector pattern is a zoom-in vector pattern or a zoom-out vector pattern.

12. The apparatus of claim 11, wherein the zoom-in vector pattern comprises a first zoom-in vector pattern and a second zoom-in vector pattern pointing in a direction from a circle center to a circumference, and the zoom-out vector pattern comprises a first zoom-out vector pattern and a second zoom-out vector pattern pointing in a direction from a circumference to a circle center.

13. The apparatus of claim 12, wherein a first zoom-in part adjusting line is formed in an extension direction of the first zoom-in vector pattern, a second zoom-in part adjusting line is formed in an extension direction of the second zoom-in vector pattern, the first zoom-in part adjusting line and the second zoom-in part adjusting line individually cross with the contour at two intersection points, respectively, and the part of the contour between the two intersection points is zoomed in in a proportion based on the shape of the original contour.

14. The apparatus of claim 12, wherein a first zoom-out part adjusting line is formed in an extension direction of the first zoom-out vector pattern, a second zoom-out part adjusting line is formed in an extension direction of the second zoom-out vector pattern, the first zoom-out part adjusting line and the second zoom-out part adjusting line individually cross with the contour at two intersection points, respectively, and the part of the contour between the two intersection points is zoomed out in a proportion based on the shape of the original contour.

15. The apparatus of claim 12, wherein when the first zoom-in vector pattern and the second zoom-in vector pattern of the zoom-in vector pattern are overlapped with each other, the scaling tool zooms in the contour in all directions.

16. The apparatus of claim 12, wherein when the first zoom-out vector pattern and the second zoom-out vector pattern of the zoom-out vector pattern are overlapped with each other, the scaling tool zooms out the contour in all directions.

17. The apparatus of claim 12, wherein the zoom-in vector pattern further comprises a third zoom-in vector pattern pointing in a direction from a circle center to a circumference, and the zoom-out vector pattern further comprises a third zoom-out vector pattern pointing in a direction from a circumference to a circle center.

18. The apparatus of claim 17, wherein the third zoom-in vector pattern is configured with a zoom-in position designating symbol, the zoom-in position designating symbol moves in an extension direction of the third zoom-in vector pattern, and the part or all of the contour being zoomed in passes through a point designated by the zoom-in position designating symbol.

19. The apparatus of claim 17, wherein the third zoom-out vector pattern is configured with a zoom-out position designating symbol, the zoom-out position designating symbol moves in an extension direction of the third zoom-out vector pattern, and the part or all of the contour being zoomed in passes through a point designated by the zoom-out position designating symbol.

20. The apparatus of claim 9, wherein the scaling tool is operated based on the cursor.
